(19) United States
(12) Patent Application Publication
(10) Pub. No.: US 2026/0248391 A1
(43) Pub. Date: Aug. 27, 2026

(54) LASER SPECKLE CONTRAST IMAGING SYSTEM AND APPLICATIONS OF SAME

(71) Applicant: VANDERBILT UNIVERSITY, Nashville, TN (US)

(72) Inventors: Emmanuel A. Mannoh, Nashville, TN (US); Anita Mahadevan-Jansen, Nashville, TN (US); Han Dong, Nashville, TN (US)

(21) Appl. No.: 18/577,298

(22) PCT Filed: Jul. 11, 2022
(86) PCT No.: PCT/US2022/036633

§ 371 Date: Jan. 8, 2024

Related U.S. Application Data
(63) Continuation-in-part of application No. 17/289,323, filed on Nov. 4, 2019, now Pat. No. 12,507,944.
Application No. PCT/US2022/036633 is a continuation-in-part of application No. 16/471,049, filed on Dec. 29, 2017, now Pat. No. 11,589,801.
(60) Provisional application No. 62/440,112, filed on Dec. 29, 2016.
(60) Provisional application No. 62/540,767, filed on Aug. 3, 2017.
(60) Provisional application No. 62/754,717, filed on Nov. 2, 2018.
(60) Provisional application No. 63/219,886, filed on Jul. 9, 2021.

(51) Int. Cl. A61B 5/00; A61B 5/026

(52) U.S. Cl. CPC A61B 5/0077; A61B 5/026; A61B 5/4227; A61B 5/742

(57) ABSTRACT

An imaging system for viability assessment of a tissue of a living subject includes a light source for emitting a beam of light; a light delivery member coupled to the light source for delivering the beam of light onto a tissue surface of the living subject to illuminate the tissue surface; a light collection member configured to collect light from the illuminated tissue surface responsive to the illumination; a detector coupled to the collection member for acquiring images of the light from the illuminated tissue surface; and a controller arranged to operate the detector to acquire the images of the light from the illuminated tissue surface, receive the acquired images from the detector, and process the acquired images t for the intraoperative assessment of tissue viability.

### CROSS-REFERENCE TO RELATED PATENT APPLICATIONS

This PCT application claims priority to and the benefit of U.S. Provisional Patent Application Ser. No. 63/219,886, filed Jul. 9, 2021.

This PCT application is also a continuation-in-part application of U.S. application Ser. No. 17/289,323, filed Apr. 28, 2021, which is a national stage entry of PCT Application Serial No. Entry of PCT/US2019/059610, filed Nov. 4, 2019, which itself claims priority to and the benefit of U.S. Provisional Patent Application Ser. No. 62/754,717, filed Nov. 2, 2018.

This PCT application is also a continuation-in-part application of U.S. application Ser. No. 16/471,049, filed Jun. 19, 2019, which is a national stage entry of PCT Application Serial No. PCT/US2017/068907, filed Dec. 29, 2017, which itself claims priority to and the benefit of U.S. Provisional Patent Application Ser. Nos. 62/440,112 and 62/540,767, filed Dec. 29, 2016 and Aug. 3, 2017, respectively.

Each of the above-identified applications is incorporated herein by reference in its entirety, respectively.

### STATEMENT AS TO RIGHTS UNDER FEDERALLY-SPONSORED RESEARCH

This invention was made with government support under Grant No. R01CA212147 awarded by the National Institutes of Health. The government has certain rights in the invention.

### FIELD OF THE INVENTION

The invention relates generally to optical assessments of bio-objects, and more particularly, to a laser speckle contrast imaging (LSCI) system that endures motion artefacts to enable intra-operative parathyroid identification and viability of assessment and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

The endocrine system is a complex system of organs and glands which includes the thyroid and parathyroid. The anatomy of the neck is illustrated in FIG. 1. The thyroid gland regulates many developmental and metabolic processes. Common diseases of the thyroid include goiters, hyperthyroidism, hypothyroidism, benign and malignant nodules, and autoimmune diseases such as Graves' disease. Surgery is the most common treatment for Graves's disease, goiters, benign thyroid nodules, and thyroid cancers.

The parathyroid normally lies within the same region as the thyroid in the neck and functions to control calcium levels in the blood. The most common parathyroid disorder is primary hyperparathyroidism, in which one or more of the parathyroid glands become enlarged and hyperactive. This causes excess secretion of parathyroid hormone and a disruption in normal bone and mineral metabolism. The prevalence of primary hyperparathyroidism has been estimated at 21 cases per 100,000 person-years. In 80% of cases, primary hyperparathyroidism is caused by a single overactive parathyroid gland and surgical removal of the diseased parathyroid gland is the only definitive treatment.

Typically, there are four tan parathyroid glands, each approximately 6 to 8 mm in size. They are typically positioned within the neck but can vary in location within the body and are sometimes intra-thymic. Due to their small size and variability in position, the parathyroid glands and their blood supplies are often difficult to distinguish from surrounding tissue and thyroid in the neck. The parathyroid visually resemble its surrounding tissue and this can extend surgical time during thyroid and parathyroid related surgeries, during which the surgeon is need to locate the small organs and their blood supply. Accidental removal or damage to the healthy parathyroid and its blood supply during surgery can result in serious postsurgical complications such as hypocalcemia and hypoparathyroidism. Hypoparathyroidism may result from direct injury, devascularization, and/or disruption of the parathyroid glands. Statistics suggest that temporary and permanent hypoparathyroidism rates are between 4-20% during thyroid surgery. The most common complications of both parathyroid and thyroid surgery are postoperative hypocalcemia, vocal-cord paralysis, and hematoma.

The current surgical procedure for thyroid and parathyroid surgeries involves a systematic search within the neck and relying on visual inspection to assess parathyroids' viability. Other methods such as lidocaine solution bathing and ICG are avoided due to their potential side effects. The incidence of complications occurring due to this subjective method is directly proportional to the extent of thyroidectomy and inversely proportional to the experience of the surgeon. The disadvantages to the current method include the lengthy duration of the surgery, the exploratory and empirical nature of the surgery, and the lack of sensitive and applicable preoperative and intra-operative imaging. Confirmation of removal of the diseased parathyroid relies on histopathology or post-operative diagnosis of symptoms. Thus, a reliable method for measure vasculature and confirm viability of the parathyroid glands is just as important as identifying these glands intraoperatively. Moreover, while surgical guidance systems have been developed and utilized for brain surgery and other organ surgery procedures, none is available for thyroid and parathyroid surgeries.

Therefore, a heretofore unaddressed need exists in the art to address the aforementioned deficiencies and inadequacies.

### SUMMARY OF THE INVENTION

In view of the foregoing, one of the objectives of this invention is to provide a portable, miniaturized imaging system capable of performing LSCI measurements intraoperatively for intraoperative assessment of parathyroid gland vascularity and reducing motion artifact introduced to the image during the intraoperative LSCI measurements. The LSCI system is developed to guide surgeons performing thyroid and parathyroid surgeries, which allows a surgeon to objectively assess a parathyroid gland's viability with LSCI during surgery. It should be appreciated that the LSCI system can be applied to not only endocrine tissues such as thyroid or parathyroid but also other tissues such as pancreas gland or adrenal gland.

In one aspect of the invention, the imaging system comprises a light source for emitting a beam of light; a light delivery member coupled to the light source for delivering the beam of light onto a tissue surface of the living subject to illuminate the tissue surface; a light collection member configured to collect light from the illuminated tissue surface responsive to the illumination; a detector coupled to the collection member for acquiring images of the light from the illuminated tissue surface, wherein each of the acquired images comprises a speckle pattern; and a controller arranged to operate the detector to acquire the images of the light from the illuminated tissue surface, receive the acquired images from the detector, and process the acquired images to obtain speckle contrast images for the intraoperative assessment of tissue viability.

In one embodiment, the light source comprises a laser.

In one embodiment, the light source comprises an infrared laser.

In one embodiment, the light source comprises a diode laser emitting the beam of light at a wavelength of about 785 nm.

In one embodiment, the light delivery member comprises at least one optical fiber optically coupled to the light source for delivering the beam of light emitted from the light source.

In one embodiment, a diffuser placed at a distal end of the at least one optical fiber for diffusing the beam of light onto the tissue surface.

In one embodiment, the diffuser is a hollow center diffuser.

In one embodiment, a beam shaper is placed at a distal end of the at least one optical fiber for focusing or collimating or directing the beam of light onto the tissue surface.

In one embodiment, the beam shaper comprises a biconvex lens.

In one embodiment, the beam shaper comprises a ball lens.

In one embodiment, the light delivery member further comprises a linear polarizer placed at the front of the optic.

In one embodiment, the collection member comprises one or more optical fibers, including a fiber bundle.

In one embodiment, the collection member further comprises a rod lens placed on a distal end of the one or more optical fibers to optimize the collection of light and image formation at a predetermined working distance.

In one embodiment, the collection member further comprises a polarizer placed on a distal end of the one or more optical fibers for reduction of spectral reflection.

In one embodiment, the system further comprises at least one lens placed in an optical path between the collection member and the detector for collecting and focusing light from the one or more optical fibers onto the detector.

In one embodiment, the detector comprises at least one camera.

In one embodiment, a high-speed camera is deployed for motion error control. In one embodiment, a camera that is capable of capturing moving images with exposures of less than 1/20 second or frame rates in excess of 20 fps is deployed for motion error control.

In one embodiment, the at least one camera comprises an infrared camera or a near-infrared camera.

In one embodiment, the at least one camera comprises a charge-coupled device (CCD) camera or a complementary metal oxide semiconductor (CMOS) camera.

In one embodiment, the system further comprises a display for displaying the speckle contrast images of the tissue in real-time.

In one embodiment, in the speckle contrast images, a perfused parathyroid gland has low speckle contrast, and a devascularized parathyroid gland has high speckle contrast.

In one embodiment, the tissue is a parathyroid gland, a pancreas gland, or an adrenal gland.

In another aspect, the invention relates to a system for assessment of a tissue of a living subject, comprising a light source for emitting a beam of light to illuminate the tissue; and an imaging head positioned over the tissue for acquiring laser speckle contrast imaging (LSCI) images of light from the illuminated tissue responsive to the illumination.

In one embodiment, the light source comprises an infrared laser.

In one embodiment, the imaging head comprises: an illumination channel coupled to the light source for delivering the beam of light onto the tissue surface to illuminate the tissue surface; and an imaging channel positioned in relationship to the illumination channel for acquiring images of the light from the illuminated tissue surface, wherein each of the acquired images comprises a speckle pattern.

In one embodiment, the light source is placed in the illumination channel.

In one embodiment, a diffuser is placed at a distal end of the illumination channel for diffusing the beam of light onto the tissue surface.

In one embodiment, a beam shaper is placed at a distal end of the illumination channel for focusing or collimating or directing the beam of light onto the tissue surface.

In one embodiment, the beam shaper comprises a ball lens.

In one embodiment, the illumination channel further comprises a linear polarizer placed at the front of the optic.

In one embodiment, the imaging channel comprises at least one camera.

In one embodiment, the at least one camera comprises a charge-coupled device (CCD) camera or a complementary metal oxide semiconductor (CMOS) camera.

In one embodiment, the imaging channel further comprises a polarizer placed the front of the at least one camera for reduction of spectral reflection.

In one embodiment, the imaging channel is 1.5 mm in diameter.

In one embodiment, the system further comprises a controller configured to control operations of the imaging head for acquiring the LSCI images of the illuminated tissue, receiving the acquired LSCI images from the at least one camera, and processing the acquired LSCI images to obtain speckle contrast images for the intraoperative assessment of tissue viability.

In yet another aspect, the invention relates to a method for assessment of a tissue of a living subject, comprising: directing a beam of light onto a tissue surface to illuminate the tissue surface; acquiring laser speckle contrast imaging (LSCI) images of light from the illuminated tissue responsive to the illumination; and processing the acquired LSCI images for the intraoperative assessment of tissue viability.

In one embodiment, said processing the acquired images comprises calculating a plurality of speckle contrasts from the acquired images of the tissue.

In one embodiment, said calculating plurality of speckle contrasts comprises: defining a window with a number of pixels over which a speckle contrast is to be calculated; moving the window across the acquired image of the speckle pattern; and at each location, calculating the speckle contrast as a standard deviation of pixel intensity values σₛ within the window divided by a mean intensity value [symbol] I [symbol] as follows:

$${K}_{s} = \frac{{σ}_{s}}{〈 I 〉}$$

wherein the resultant speckle contrast image has values that range from 0 to 1, with values closer to 0 representing regions of greater motion (perfusion) and 1 representing regions with no motion.

In one embodiment, said processing the acquired images comprises an LSCI threshold mechanism for frame selection that removes frames that contain excessive motion, thereby realizing the intraoperative assessment of tissue viability.

In one embodiment, a frame selection code is applied to selection frames with tolerable motion.

In one embodiment, a universal mean of the contrast value over each frame is threshold with a calculated number based on the acquired video and threshold applied, such that when a frame is below the threshold, there exists excessive amount of motion artifact that confounds with the blood flow information in the frame; and when a frame are above the threshold, the frame is acquired in relative stillness and the motion artifact that is introduced during the exposure time is tolerable by the LSCI.

In one embodiment, said processing the acquired images comprises discarding a frame out of the video when the frame is below the threshold, and preserving a frame when the frame is above the threshold and using the preserved frame an indicator of whether the target of interest is well vascularized.

In one embodiment, a machine learning algorithm is applied to reduce motion artifact on the acquired LSCI images.

In one embodiment, the method further comprises displaying the speckle contrast images of the tissue in real-time.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein. The drawings described below are for illustration purposes only. The drawings are not intended to limit the scope of the present teachings in any way.

FIG. 1 shows a general view of the anatomy of human thyroid/parathyroid glands.

FIG. 2 shows schematically a laser speckle contrast imaging (LSCI) system according to embodiments of the invention.

FIG. 3 shows schematically an LSCI system according to embodiments of the invention.

FIG. 4 shows schematically an LSCI system according to embodiments of the invention.

FIG. 5 shows schematically an LSCI system according to embodiments of the invention.

FIGS. 6A-6C show schematically an LSCI system according to embodiments of the invention. FIG. 6A: a perspective view; FIG. 6B: a transparent view; and FIG. 6C: a lateral cross section.

FIG. 7 shows a flowchart of automated image processing according to embodiments of the invention. Automated imaging processing for laser speckle contrast imaging can be done in many other ways. The flow chart shows one way to overcome the movement artifact that are introduced by the device being handheld. In the exemplary embodiment of the process, a user needs to start the laser for speckle illumination and the camera for image streaming; aim the camera at the target parathyroid at a distance that the camera can be in focus, which one can either use computer for focus check or just via a display connected to the computer with human eyes; an automated program runs and checks for the optimum exposure time for laser speckle contrast imaging; with the optimum exposure time determined, the camera starts to acquire a speckle video. Once the video is taken or during the acquisition, the computer processes the raw video to a laser speckle contrasting video. Another program is ran to determine a threshold of the contrast value. The threshold is then applied to the laser speckle contrast video that only frames that are held rather stationary remain. A screen that connects to the computer displays the selected frames. Subsequently, about 10 successive selected frames are averaged for their area of interest's average contrast value and finally, a value for the parathyroid speckle contrast is obtained and fitted to a logistic regression model to determine the likelihood of devascularization. Thus it acts as an aiding tool for the surgeons to determine the vascularity/viability of the parathyroids or other organs.

FIG. 8A-8B show phantom data according to embodiments of the invention. In general this is one embody of color hue that blue indicate motion of the object in the image and yellow represent static of that. However, the color choice does not affect the result of laser speckle contrast imaging. For LSCI, in general the lower the value of the pixel (contrast value) corresponds to the faster in motion and vice versa. The contrast value can be converted into the speed of the flow (blood flow in this case). Thus, the LSCI can be converted into a speed map. FIG. 8A: Indicating that there are flow in the blood vessel phantom. FIG. 5B: Indicating that there are no flow in the blood vessel phantom.

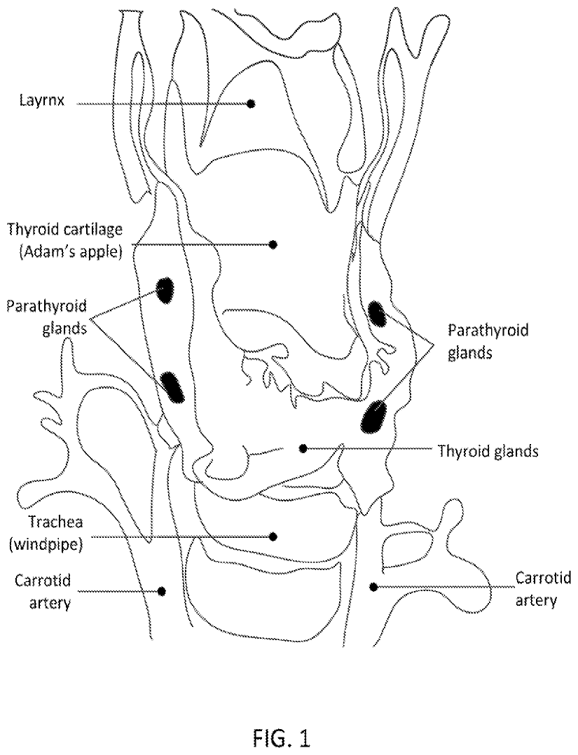

### DETAILED DESCRIPTION OF THE INVENTION

The present invention will now be described more fully hereinafter with reference to the accompanying drawings, in which exemplary embodiments of the present invention are shown. The present invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the present invention.

It will be understood that, as used in the description herein and throughout the claims that follow, the meaning of “a”, “an”, and “the” includes plural reference unless the context clearly dictates otherwise. Also, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this disclosure, the phrase “at least one of A, B, and C” should be construed to mean a logical (A or B or C), using a non-exclusive logical OR. As used herein, the term “and/or” includes any and all combinations of one or more of the associated listed items.

As used in this disclosure, the term “living subject” refers to a human being such as a patient, or a mammal animal such as a monkey.

As used in this disclosure, “charge-coupled device” or “CCD” refers to an analog shift register that enables the transportation of analog signals (electric charges) through successive stages (capacitors), controlled by a clock signal. Charge-coupled devices can be used as a form of memory or for delaying samples of analog signals. Today, they are most widely used in arrays of photoelectric light sensors to serialize parallel analog signals. In a CCD for capturing images, there is a photoactive region (an epitaxial layer of silicon), and a transmission region made out of a shift register (the CCD, properly speaking).

An image is projected through a lens onto the capacitor array (the photoactive region), causing each capacitor to accumulate an electric charge proportional to the light intensity at that location. A one-dimensional array, used in line-scan cameras, captures a single slice of the image, while a two-dimensional array, used in video and still cameras, captures a two-dimensional picture corresponding to the scene projected onto the focal plane of the sensor. Once the array has been exposed to the image, a control circuit causes each capacitor to transfer its contents to its neighbor (operating as a shift register). The last capacitor in the array dumps its charge into a charge amplifier, which converts the charge into a voltage. By repeating this process, the controlling circuit converts the entire semiconductor contents of the array to a sequence of voltages, which it samples, digitizes and stores in some form of memory.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Laser speckle contrast imaging (LSCI) is a widefield imaging technique capable of assessing superficial blood flow of parathyroid gland vascularity during endocrine surgery, which utilizes intrinsic tissue contrast from dynamic light scattering and provides a relatively simple technique for visualizing detailed spatiotemporal dynamics of blood flow changes in real-time. Laser speckle is the random interference pattern produced when coherent light scatters from a random medium and can be imaged onto a detector. Motion from scattering particles, such as red blood cells in the vasculature, leads to spatial and temporal variations in the speckle pattern. Speckle contrast analysis quantifies the local spatial variance, or blurring, of the speckle pattern that results from blood flow. Areas with greater motion have more rapid intensity fluctuations and therefore have more blurring of the speckles during the camera exposure time. LSCI can be used to quantify relative changes in blood flow.

The LSCI technique analyzes the interference pattern produced when coherent light is incident on a surface. Minute differences in path length created by the light waves scattering from different regions of the surface produce bright and dark spots of constructive and destructive interference respectively, termed as a speckle pattern. This speckle pattern fluctuates depending on how fast particles are moving within a few microns of the surface. Blurring of the speckle pattern occurs when the motion is fast relative to the integration time of the detector. Analyzing this spatial blurring provides contrast between regions of faster versus slower motion and forms the basis of LSCI. This technique is sensitive to microvascular perfusion and has been employed in a variety of tissues where the vessels of interest are generally superficial, such as the retina, skin and brain. Parathyroid glands are densely packed with blood vessels, given that they secrete PTH to the entire body. Furthermore, their small size (3-8 mm) makes many of these vessels superficial, making these glands suitable targets for assessment using LSCI. Certain aspects of this invention disclose a combined auto-fluorescence and LSCI system for assessment of parathyroid gland vascularity during endocrine surgery.

The inventors have disclosed in U.S. patent application Ser. Nos. 16/471,049 and 17/289,323, which are incorporated herein by reference in their entireties, that LSCI is an effective method for evaluating blood flow in parathyroid glands, and measurements made by this technique are strongly related to the postoperative outcomes of total thyroidectomy patients. However, the previous work involved a static imaging device that is not always ideal for small surgical incisions and to image hard-to-reach parathyroid glands.

In view of the foregoing, one of the objectives of this invention is to provide a portable, miniaturized imaging system capable of performing LSCI measurements intraoperatively for intraoperative assessment of parathyroid gland vascularity and reducing motion artifact introduced to the image during the intraoperative LSCI measurements. The LSCI system is developed to guide surgeons performing thyroid and parathyroid surgeries, which allows a surgeon to objectively assess a parathyroid gland's viability with LSCI during surgery. It should be appreciated that the LSCI system can be applied to not only endocrine tissues such as thyroid or parathyroid but also other tissues such as pancreas gland or adrenal gland. In addition to laser speckle contrast imaging system, the invention can also be applied to other motion sensitive optical systems that may suffer from motion artefacts, such as laser Doppler imaging (LDI), multi-spectral laser speckle contrast images, speckle interferometry, etc.

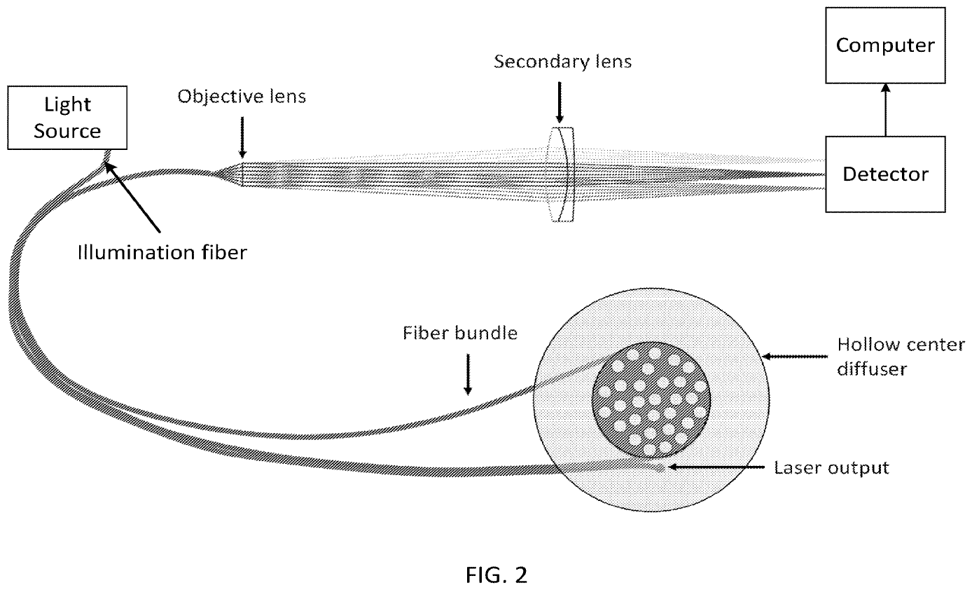

Referring to FIG. 2, the LSCI system in one exemplary embodiment includes an imaging fiber bundle, which is used to relay images from a surgical site to a detector. The end of the fiber bundle at the surgical site is referred to as the distal end, and the end connected to the detector is the proximal end. In one embodiment, a rod lens is placed on the distal end of the fiber bundle to optimize the collection of light and image formation at the chosen working distance. At the proximal end, an objective lens is adapted to collect light from the fiber bundle and image it through a secondary lens or system of lenses onto the detector. The lenses are chosen to magnify the image from the distal end of the fiber bundle to fill the detector. For example, as shown in FIG. 2, a 20× objective (the objective lens) is paired with an 80 mm focal length converging lens (the secondary lens) to magnify the image from a 1 mm diameter fiber bundle onto an 8.7 mm diameter CMOS sensor (the detector). To provide illumination for speckle imaging, an optical fiber is connected to a light source of a laser module of any wavelength and is run alongside the fiber bundle. In some embodiments, the light source is an infrared laser, e.g., a single mode 785 nm diode laser, or the like. It should be noted that any other wavelengths can also be used to practice the invention.

In addition, a diffuser, e.g., hollow center diffuser shown in FIG. 2, is attached at the distal end of the illumination optical fiber to produce a more uniform speckle pattern on the target tissue to be imaged. In one embodiment, a beam shaper is placed at a distal end of the illumination channel for focusing or collimating or directing the beam of light onto the tissue surface. In one embodiment, the beam shaper can be a lens tube having a biconvex lens coupled to the light source for directing the beam of light onto a tissue surface of a parathyroid gland (e.g., object) of a patient to illuminate the tissue surface. In one embodiment, the beam shaper can be a ball lens.

In some embodiments, the detector includes a camera, such as an infrared camera or a near-infrared (NIR) camera. The camera can be a charge-coupled device (CCD) camera or a complementary metal-oxide-semiconductor (CMOS) camera. In some embodiments, the detector further includes a focus tunable lens (e.g., a zoom lens) attached to the front end of the camera.

In some embodiments, a high-speed camera is deployed for motion error control. In one embodiment, a camera that is capable of capturing moving images with exposures of less than 1/20 second or frame rates in excess of 20 fps is deployed for motion error control. In some embodiments, the camera is adapted to individually acquire the LSCI images.

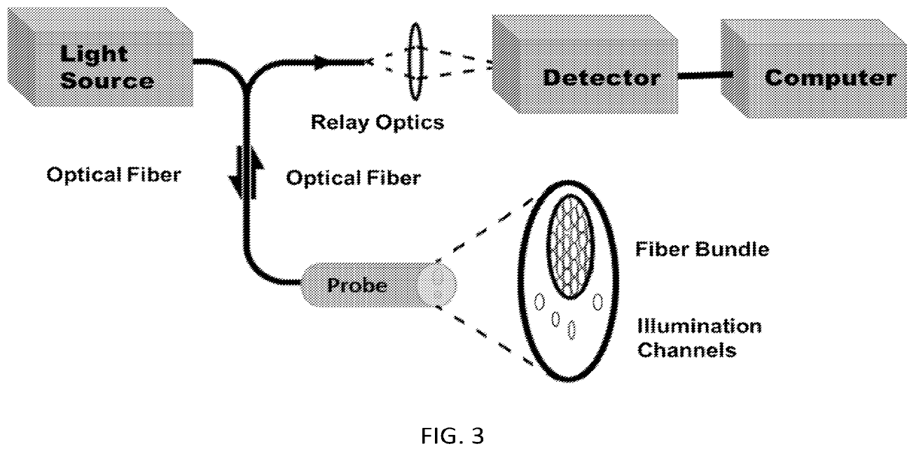

In one embodiment as shown in FIG. 3, the illumination channel and the imaging fiber bundle are bundled into one rigid or flexible probe, which can be hold by a user in the operating room. Using the probe, parathyroid glands can be more easily evaluated in cases with small surgical incisions.

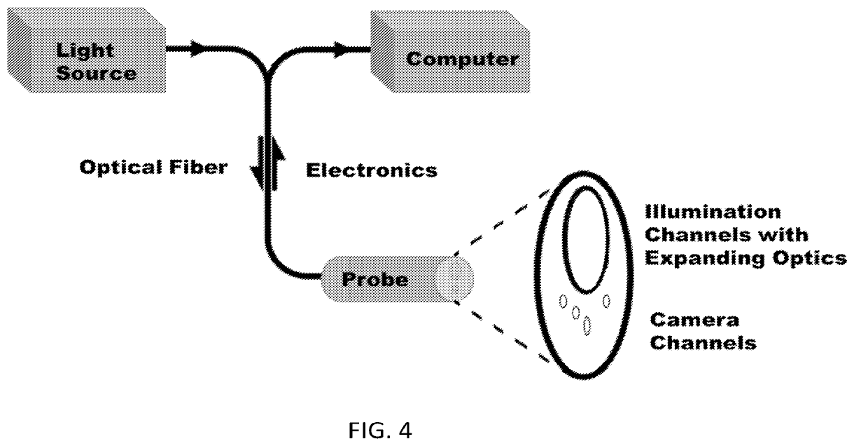

Besides an imaging fiber bundle, a micro-camera can also be employed for performing LSCI measurements. In one embodiment as shown in FIG. 4, the camera is placed at the distal end of the probe, adjacent to the illumination channel, while the wiring connecting the camera runs to the distal end outside the surgical field where it is connected to the appropriate electronics for recording images. For example, in the mini-camera system schematic, a mini-camera with 249×250 pixels is deployed at the distal end of the probe. To provide illumination for speckle imaging, an optical fiber is connected to a laser module of any wavelength and runs alongside the imaging channel. A diffuser can be attached at the distal end of the illumination optical fiber to produce a more uniform speckle pattern on the target tissue to be imaged. An optional white light fiber can be attached to the bundle for white light illumination and better focusing experience. Bundling the imaging channel, fiber optics, and the optional white light fiber into one rigid or flexible probe, parathyroid glands can be more easily evaluated in cases with small surgical incisions.

FIGS. 3-4 shows two different embodiments of the laser speckle contrast imaging system. The base laser speckle contrast system can be composed of the illumination, which in the two embodiments are the laser and optical fiber that deliver light to the region of interest, but the illumination can well be illuminated from a light source through free space and through other media. Acquisition channel can be composed of optical fiber that collects data from the region of interest or be composed of a camera upfront with electronics or wireless connections that send data back to the process. The data acquisition channel can be realized through other forms. In addition, it is also possible to contain the laser component and/or camera component and/or computational component and/or display component into the probe as a wire/wireless device.

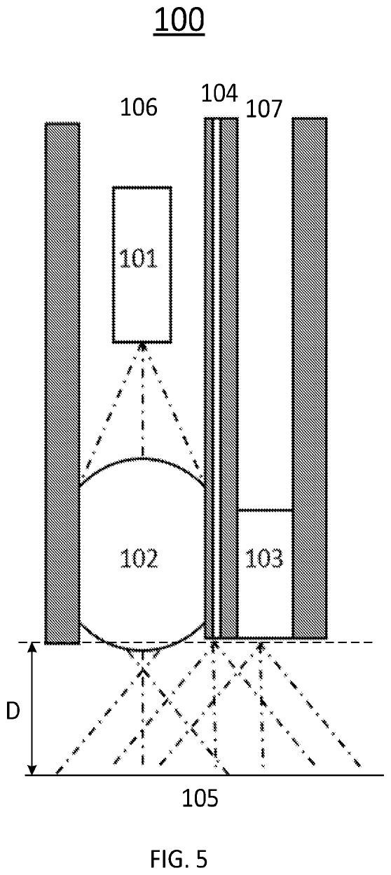

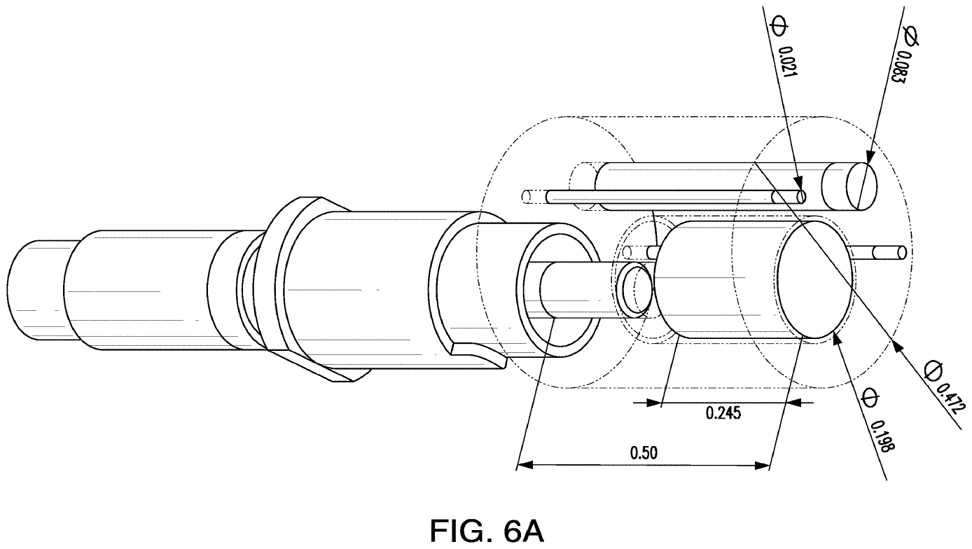

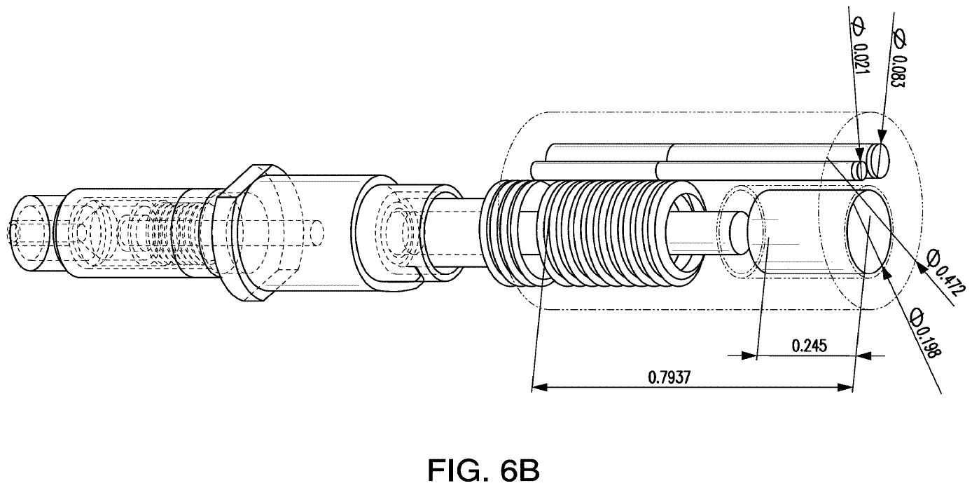

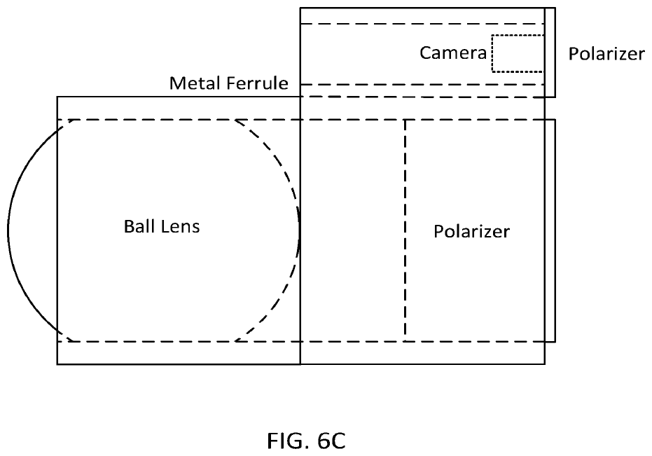

Referring to FIGS. 5 and 6A-6C, and particularly to FIG. 5, the handheld LSCI system (i.e., the probe) 100 is shown according to embodiments of the invention. The LSCI system 100 includes a light source channel (i.e., illumination channel) 106 for emitting a beam of light to illuminate a target of interest 105; and an imaging channel 107 positioned next to the light source channel 106 aiming at the target of interest 105 for acquiring LSCI images of light from the illuminated target of interest 105 responsive to the illumination. In some embodiments, the imaging channel 107 is about 1.5 mm in diameter.

In certain embodiments, the light source is, but is not limited to, an infrared laser. The infrared laser is a diode laser emitting a beam of light at a wavelength of about 785 nm.

In certain embodiments, the imaging channel 107 comprises a detector 103 with a metal ferrule for individually acquiring the LSCI images. The detector 103 such as a camera is adapted for collecting the light from the illuminated target of interest 105 in a surgical field.

In certain embodiments, the distance, D, between the distal end of the probe 100 and the target of interest 105 is about 2 cm. The choice of about 2 cm working distance is to keep the imaging probe relatively compact and friendly to use. It should be noted that such a distance D can be adjusted by refocusing with optical instrument.

In certain embodiments, a short focal length lens 102 is deployed in the front of the illumination fiber 101 to match the field of view of that of the detector 103. In one embodiment, a sapphire ball lens is deployed due to its power and compactness. In some embodiments, an alternative to the optical fiber 101 for illumination is a small laser diode placed at the distal end of the probe in a relationship to the ball lens 102 such that the ball lens 102 is between the target of interest 105 and the small laser diode 101.

In certain embodiments, channels of fibers 104 are installed for white light illumination pre-LSCI image acquisition. Such a design helps the user with the orientation of the device and image capturing.

In certain embodiments, as shown in FIG. 6C, polarizers are deployed in the front of the illumination channel and the imaging channel either separately or together to reduce the effect of spectral reflection. In some embodiments, a polarizer is placed in the front of the camera perpendicular to the polarization orientation of the illumination channel to reduce spectral reflection. In one embodiment, a linear polarizer is positioned in the front of the illumination.

FIGS. 5 and 6A-6C show the exemplary embodiments of the image system according to the invention, the imaging system is enclosed in a sealed and sterilizable probe that can be hold by the user in the operating room and reusable afterwards. In the exemplary embodiments, on the interface end of the probe is the imaging head, where imaging channel contains commercial camera Naneye (AMS, Premstaetten, Austria) for LSCI image acquisition as well as white light image acquisition. Illumination channel contains single mode fiber with FC/PC connector (Thorlab, Newton, NJ) and a 6 mm diameter sapphire ball lens (Edmund Optics, Barrington, NJ), 5 mm apart from each other. Together, the illumination channel can illuminated the region of interested with good quality speckle pattern illumination that can be easily picked up by the built-in camera and thus analyzed by the computer mentioned previously.

In addition, as shown in FIGS. 2-4, the LSCI system further comprises a controller (alternative computer) configured to control operations of the imaging head (alternative the probe) for acquiring the LSCI images of the illuminated target of interest, receiving the acquired LSCI images from the detector, and processing the acquired LSCI images to obtain speckle contrast images for the intraoperative assessment of parathyroid gland viability. In one embodiment, in the speckle contrast images, a perfused parathyroid gland has low speckle contrast, and a devascularized parathyroid gland has high speckle contrast. In one exemplary embodiment, on the proximal end of the probe sits the computer that controls the instrument (home-built machine with six 3.7 GHz cores-Intel OEM Core i7-8700K) and a single mode 785 nm diode laser with 80 mW power output (Innovative Photonics Solutions, Monmouth Junction, NJ), guided through electronics and optical fiber respectively.

Further, the system may also include a display for displaying the speckle contrast images of the parathyroid gland in real-time.

Conventional laser speckle contrast imager appears less ergonomically suitable (more bulky) for use in the OR in the assessment of parathyroid glands than the invented handheld LSCI device. According to the invention, the handheld LSCI device can be combined with another lab-built device for detecting parathyroid gland using fluorescence, and marketed to endocrine surgeons.

In certain aspects of the invention, the handheld LSCI system can very feasibly be combined with near-infrared autofluorescence detection, which is used for intraoperative parathyroid identification. The result of such a combination would provide surgeons with a single tool to both identify parathyroid glands and assess their vascularity during operations. This has the potential to improve patient outcomes after surgery. There is currently one FDA cleared clinical device for intraoperative parathyroid detection using near-infrared autofluorescence detection. Called the PTEye, this device originated from a collaboration between the original manufacturing company AiBiomed, and the inventor Dr. Mahadevan-Jansen. Extending the PTEye's capabilities to include parathyroid vascularity assessment could involve attaching a fiber bundle to the already existing probe and a camera and lenses to image the output of the fiber bundle as described above. The laser source used in the PTEye for autofluorescence detection could also be used for laser speckle contrast imaging.

In addition, the handheld LSCI device/probe according to the invention allows for a more compact final product capable of assisting surgeons in first identifying parathyroid glands, and then assessing their viability.

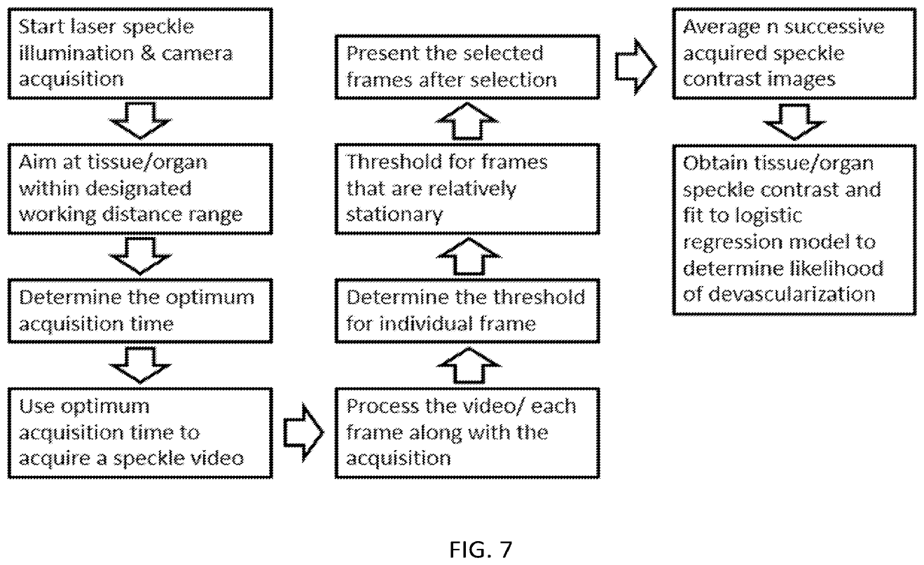

In certain aspects, the invention relates a method for intraoperative assessment of parathyroid gland viability of a living subject for guidance in a surgery. FIG. 7 shows schematically a flowchart for intraoperative assessment of parathyroid gland viability of a living subject according to one embodiment of the invention. The method includes providing a beam of light onto a tissue surface of a parathyroid gland of a patient to illuminate the tissue surface; acquiring images of the illuminated tissue surface, where each of the acquired images comprises a speckle pattern; and processing the acquired images to obtain speckle contrast images for the intraoperative assessment of parathyroid gland viability. In the speckle contrast images, a perfused parathyroid gland has low speckle contrast, and a devascularized parathyroid gland has high speckle contrast.

In some embodiments, said processing the acquired images of the parathyroid gland by the controller is performed with calculating a plurality of speckle contrasts from the acquired images of the parathyroid gland.

In some embodiments, said calculating plurality of speckle contrasts comprises defining a window with a number of pixels over which a speckle contrast is to be calculated; moving the window across the acquired image of the speckle pattern; and at each location, calculating the speckle contrast as a standard deviation of pixel intensity values σₛ within the window divided by a mean intensity value [symbol] I [symbol] as follows:

$${K}_{s} = \frac{{σ}_{s}}{〈 I 〉}$$

where the resultant speckle contrast image has values that range from 0 to 1, with values closer to 0 representing regions of greater motion (perfusion) and 1 representing regions with no motion.

In some embodiments, said processing the acquired LSCI images comprises an LSCI threshold mechanism for frame selection that gets rid of the frames that contain excessive motion and thus realizes assessment of the viability of the target parathyroid intraoperatively. In some embodiments, a frame selection code is applied to selection frames with tolerable motion. In some embodiments, a machine learning algorithm is applied to reduce the motion artifact have on the LSCI taken by the system.

In some embodiments, the algorithm developed to automatically threshold for motion artifact that is introduced into the captured frame. A universal mean of the contrast value over the each frame is threshold with a calculated number based on the taken video and threshold applied. Any frame that is below the threshold means that there exist excessive amount of motion artifact that will confound with the blood flow information and thus will be discarded out of the video; in contrary, frames that are above the threshold means that the frame was taken in relative stillness and the motion artifact that was introduced during the exposure time is tolerable by the laser speckle contrast image and thus should be preserved and used as indicator of whether the parathyroid is well vascularized.

Specifically, as shown in FIG. 7, in the exemplary embodiment of the process, a user needs to start the laser for speckle illumination and the camera for image streaming; aim the camera at the target parathyroid at a distance that the camera can be in focus, which one can either use computer for focus check or just via a display connected to the computer with human eyes; an automated program runs and checks for the optimum exposure time for laser speckle contrast imaging; with the optimum exposure time determined, the camera starts to acquire a speckle video. Once the video is taken or during the acquisition, the computer processes the raw video to a laser speckle contrasting video. Another program is ran to determine a threshold of the contrast value. The threshold is then applied to the laser speckle contrast video that only frames that are held rather stationary remain. A screen that connects to the computer displays the selected frames. Subsequently, about 10 successive selected frames are averaged for their area of interest's average contrast value and finally, a value for the parathyroid speckle contrast is obtained and fitted to a logistic regression model to determine the likelihood of devascularization. Thus it acts as an aiding tool for the surgeons to determine the vascularity/viability of the parathyroids or other organs.

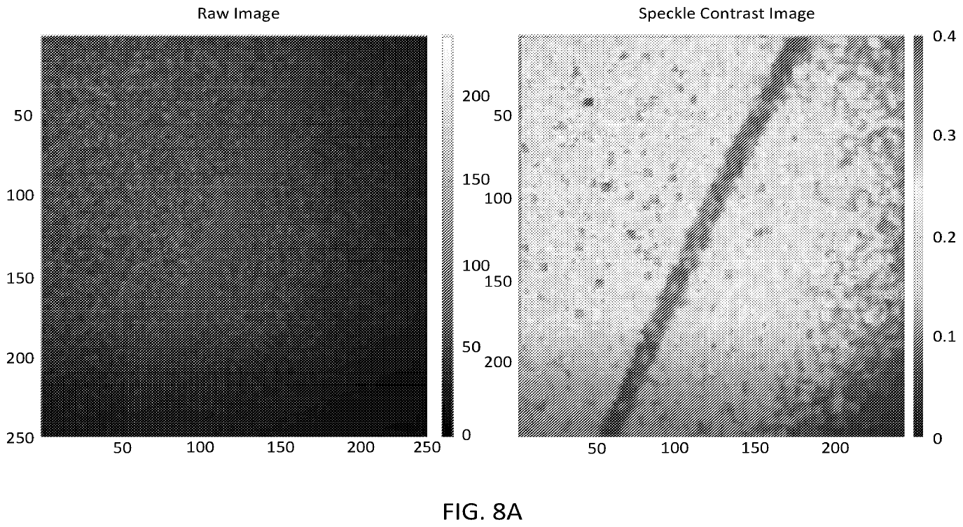

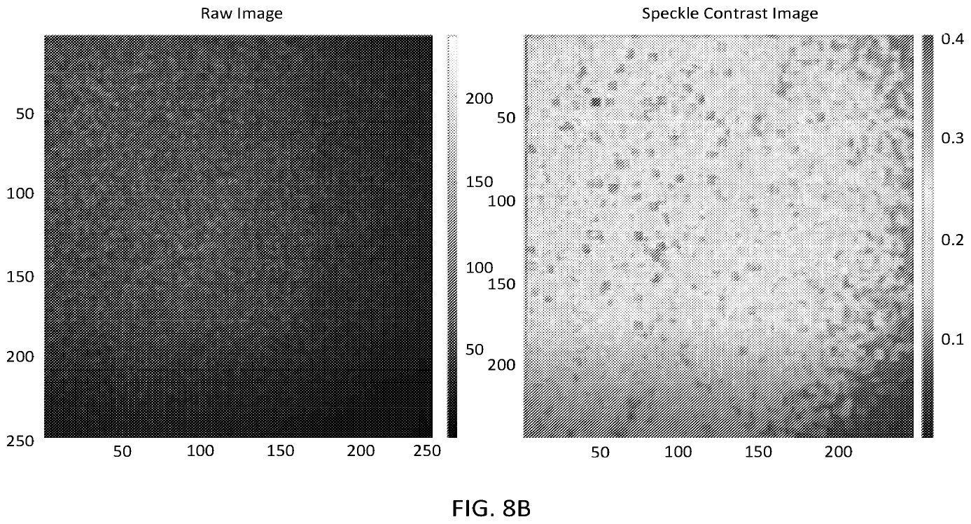

FIG. 8A-8B show phantom data according to embodiments of the invention. In general this is one embody of color hue that blue indicate motion of the object in the image and yellow represent static of that. However, the color choice does not affect the result of laser speckle contrast imaging. For LSCI, in general the lower the value of the pixel (contrast value) corresponds to the faster in motion and vice versa. The contrast value can be converted into the speed of the flow (blood flow in this case). Thus, the LSCI can be converted into a speed map.

In certain aspects, this invention uses laser speckle imaging to provide a real-time non-invasive means to inform the surgeons whether or not a parathyroid gland is still being perfused and is therefore viable. During a thyroid or parathyroid surgery, the invented apparatus or device acquires and processes images of the parathyroid gland. The device comprises a 785 nm wavelength laser and a near-infrared camera with a zoom lens, positioned above the surgical filed via an articulated arm. The laser light is diffused onto the tissue surface and images are acquired by the camera. Images are acquired using a program developed on commercially available software, and processed to produce speckle contrast image through the lab-developed dynamic link library file written in C++. This allows the speckle contrast images to be displayed to the surgeon in real-time (as they are acquired). In these speckle contrast images, perfused (and therefore viable) parathyroid glands generally have low speckle contrast, while devascularized parathyroid glands have high speckle contrast.

Without intent to limit the scope of the invention, some exemplary embodiments are given below:

In one aspect of the invention, the imaging system for assessment of a tissue of a living subject comprises a light source for emitting a beam of light; a light delivery member coupled to the light source for delivering and directing the beam of light onto a tissue surface of the living subject to illuminate the tissue surface; a light collection member configured to collect light from the illuminated tissue surface responsive to the illumination; a detector coupled to the collection member for acquiring images of the light from the illuminated tissue surface, wherein each of the acquired images comprises a speckle pattern; and a controller arranged to operate the detector to acquire the images of the light from the illuminated tissue surface, receive the acquired images from the detector, and process the acquired images to obtain speckle contrast images for the intraoperative assessment of tissue viability. The intraoperative assessment may also include, but is not limited to, evaluating perfusion/blood flow of the tissue. In some embodiments, the tissue is a parathyroid gland, a pancreas gland, or an adrenal gland.

In some embodiments, the light source comprises an infrared laser.

In some embodiments, the light source comprises a diode laser emitting the beam of light at a wavelength of about 785 nm. It should be noted that other wavelengths can also be used to practice the invention.

In some embodiments, the light delivery member comprises at least one optical fiber optically coupled to the light source for delivering the beam of light emitted from the light source.

In some embodiments, a diffuser placed at a distal end of the at least one optical fiber for diffusing the beam of light onto the tissue surface. The distal end of the at least one optical fiber is the end that operably close to the target of interest.

In some embodiments, the diffuser is a hollow center diffuser.

In one embodiment, a beam shaper is placed at a distal end of the illumination channel for focusing or collimating or directing the beam of light onto the tissue surface.

In some embodiments, the beam shaper comprises a biconvex lens.

In some embodiments, the beam shaper comprises a ball lens.

In some embodiments, the light delivery member further comprises a linear polarizer placed at the front of the diffuser.

In some embodiments, the collection member comprises one or more optical fibers, including a fiber bundle.

In some embodiments, the collection member further comprises a rod lens placed on a distal end of the one or more optical fibers to optimize the collection of light and image formation at a predetermined working distance. The distal end of the one or more optical fibers is the end that operably close to the target of interest.

In some embodiments, the collection member further comprises a polarizer placed on a distal end of the one or more optical fibers for reduction of spectral reflection.

In some embodiments, the system further comprises at least one lens placed in an optical path between the collection member and the detector for collecting and focusing light from the one or more optical fibers onto the detector.

In some embodiments, the detector comprises at least one camera.

In some embodiments, a high-speed camera is deployed for motion error control. In one embodiment, a camera that is capable of capturing moving images with exposures of less than 1/20 second or frame rates in excess of 20 fps is deployed for motion error control.

In some embodiments, the at least one camera comprises an infrared camera or a near-infrared camera.

In some embodiments, the at least one camera comprises a charge-coupled device (CCD) camera or a complementary metal oxide semiconductor (CMOS) camera.

In some embodiments, the system further comprises a display for displaying the speckle contrast images of the tissue in real-time.

In some embodiments, in the speckle contrast images, a perfused parathyroid gland has low speckle contrast, and a devascularized parathyroid gland has high speckle contrast.

In another aspect of the invention, the imaging system for assessment of a tissue of a living subject, comprising a light source for emitting a beam of light to illuminate the tissue; and an imaging head positioned over the tissue for acquiring laser speckle contrast imaging (LSCI) images of light from the illuminated tissue responsive to the illumination.

In some embodiments, the light source comprises an infrared laser.

In some embodiments, the imaging head comprises: an illumination channel coupled to the light source for delivering and directing the beam of light onto the tissue surface to illuminate the tissue surface; and an imaging channel positioned in relationship to the illumination channel for acquiring images of the light from the illuminated tissue surface, wherein each of the acquired images comprises a speckle pattern.

In some embodiments, the light source is placed in the illumination channel.

In some embodiments, the illumination channel comprises a diffuser placed at a distal end of the illumination channel for diffusing the beam of light onto the tissue surface. The distal end of the illumination channel is the end that operably close to the tissue.

In some embodiments, the illumination channel comprises a beam shaper placed at a distal end of the at least one optical fiber for focusing or collimating or directing the beam of light onto the tissue surface.

In some embodiments, the beam shaper comprises a ball lens.

In some embodiments, the illumination channel further comprises a linear polarizer placed at the front of the optic.

In some embodiments, the imaging channel comprises at least one camera.

In some embodiments, the at least one camera comprises a CCD camera or a CMOS camera.

In some embodiments, the imaging channel further comprises a polarizer placed the front of the at least one camera for reduction of spectral reflection.

In some embodiments, the imaging channel is 1.5 mm in diameter.

In some embodiments, the system further comprises a controller configured to control operations of the imaging head for acquiring the LSCI images of the illuminated tissue, receiving the acquired LSCI images from the at least one camera, and processing the acquired LSCI images to obtain speckle contrast images for the intraoperative assessment of tissue viability.

In yet another aspect of the invention, the method for assessment of a tissue of a living subject, comprising: directing a beam of light onto the tissue surface to illuminate the tissue surface; acquiring laser speckle contrast imaging (LSCI) images of light from the illuminated tissue responsive to the illumination; and processing the acquired LSCI images for the intraoperative assessment of tissue viability.

In some embodiments, said processing the acquired images comprises calculating a plurality of speckle contrasts from the acquired images of the tissue.

In some embodiments, said calculating plurality of speckle contrasts comprises: defining a window with a number of pixels over which a speckle contrast is to be calculated; moving the window across the acquired image of the speckle pattern; and at each location, calculating the speckle contrast as a standard deviation of pixel intensity values σₛ within the window divided by a mean intensity value [symbol] I [symbol] as follows:

$${K}_{s} = \frac{{σ}_{s}}{〈 I 〉}$$

wherein the resultant speckle contrast image has values that range from 0 to 1, with values closer to 0 representing regions of greater motion (perfusion) and 1 representing regions with no motion.

In some embodiments, said processing the acquired images comprises an LSCI threshold mechanism for frame selection that removes frames that contain excessive motion, thereby realizing the viability assessment of the target of interest intraoperatively.

In some embodiments, a frame selection code is applied to selection frames with tolerable motion. In some embodiments, a frame selection code is applied to selection frames with tolerable motion. One method of realization is that a universal threshold is applied to all frames. The threshold take the mean of all pixels of individual frames and filter out the frames with lower mean contrast value, as lower contrast value of the whole image is an indication that the frame is blurred out (contains excessive motion).

In some embodiments, a universal mean of the contrast value over each frame is threshold with a calculated number based on the acquired video and threshold applied, such that when a frame is below the threshold, there exists excessive amount of motion artifact that confounds with the blood flow information in the frame; and when a frame are above the threshold, the frame is acquired in relative stillness and the motion artifact that is introduced during the exposure time is tolerable by the LSCI.

In some embodiments, said processing the acquired images comprises discarding a frame out of the video when the frame is below the threshold, and preserving a frame when the frame is above the threshold and using the preserved frame an indicator of whether the target of interest is well vascularized.

In some embodiments, a machine learning algorithm is applied to reduce motion artifact on the acquired LSCI images. In some embodiments, methods such as Pixel2pixel could be used to reconstruct the image with motion removed. Pix2Pix GAN is a conditional GAN (cGAN). Unlike vanilla GAN which uses only real data and noise to learn and generate images, cGAN uses real data, noise as well as labels to generate images. In essence, the generator learns the mapping from the real data as well as the noise. However, this is just one machine learning method to reduce the motion artifact. It should be noted that other machine learning methods can also be utilized to practice the invention.

In some embodiments, the method further comprises displaying the speckle contrast images of the parathyroid gland in real-time.

It should be noted that all or a part of the methods according to the embodiments of the invention is implemented by hardware or a program instructing relevant hardware.

Yet another aspect of the invention provides a non-transitory computer readable storage medium/memory that stores computer executable instructions or program codes. The computer executable instructions or program codes enable a computer or a similar computing apparatus to complete various operations of the above-disclosed method for processing LSCI images for intraoperative guidance in a surgery. The storage medium/memory may include, but is not limited to, high-speed random access medium/memory such as DRAM, SRAM, DDR RAM or other random access solid state memory devices, and non-volatile memory such as one or more magnetic disk storage devices, optical disk storage devices, flash memory devices, or other non-volatile solid state storage devices.

The foregoing description of the exemplary embodiments of the present invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. Mannoh E A, Thomas G, Solórzano C C, Mahadevan-Jansen A, “Intraoperative assessment of parathyroid viability using laser speckle contrast imaging”, Sci. Rep. 7, 14798 (2017).
[2]. Mannoh E A, Thomas G, Baregamian N, Rohde S, Solórzano CC, Mahadevan-Jansen A, “Assessing intraoperative laser speckle contrast imaging of parathyroid glands in relation to thyroidectomy patient outcomes”, Thyroid (2021).
[3]. American Thyroid Association. General Information/Press Room. (2017). at <http://www.thyroid.org/media-main/about-hypothyroidism/>
[4] Sun, G. H., DeMonner, S. & Davis, M. M. Epidemiological and Economic Trends in Inpatient and Outpatient Thyroidectomy in the United States, 1996-2006. Thyroid 23, 727-733 (2013).
[5]. Rafferty, M. A. et al. Completion Thyroidectomy Versus Total Thyroidectomy: Is There a Difference in Complication Rates? An Analysis of 350 Patients. J. Am. Coll. Surg. 205, 602-607 (2007).
[6]. Schwartz, A. E. & Friedman, E. W. Preservation of the parathyroid glands in total thyroidectomy. Surg. Gynecol. Obstet. 165, 327-32 (1987).
[7]. Ritter, K., Elfenbein, D., Schneider, D. F., Chen, H. & Sippel, R. S. Hypoparathyroidism after total thyroidectomy: incidence and resolution. J. Surg. Res. 197, 348-53 (2015).
[8]. Demeester-Mirkine, N., Hooghe, L., Van Geertruyden, J. & De Maertelaer, V. Hypocalcemia after thyroidectomy. Arch. Surg. 127, 854-8 (1992).
[9]. Pattou, F. et al. Hypocalcemia following Thyroid Surgery: Incidence and Prediction of Outcome. World J. Surg 22, 718-724 (1998).
[10]. McHenry, C. R., Speroff, T., Wentworth, D. & Murphy, T. Risk factors for postthyroidectomy hypocalcemia. Surgery 116, 641-7-8 (1994).
[11]. Zahedi Niaki, N. et al. The Cost of Prolonged Hospitalization due to Postthyroidectomy Hypocalcemia: A Case-Control Study. Adv. Endocrinol. 2014, 1-4 (2014).
[12]. Naveh-Many, T. Molecular Biology of the Parathyroid. (Kluwer Academic/Plenum Publishers, 2005). at <http://web.b.ebscohost.com.proxy.library.vanderbilt.edu/ehost/ebookviewer/ebook/bmxl YmtfXzEyNTkwNF9fQU41?sid-0747ff07-a77e-43ea-9efe-aa979596f166@sessionmgr2&vid-0&format=EB&lpid=lp_x&rid=0>
[13]. Mohebati, A. & Shaha, A. R. Anatomy of thyroid and parathyroid glands and neurovascular relations. Clin. Anat. 25, 19-31 (2012).
[14]. Lo, C. Y. et al. Parathyroid Autotransplantation During Thyroidectomy. Arch. Surg. 136, 1381 (2001).
[15]. Sierra, M. et al. Prospective biochemical and scintigraphic evaluation of autografted normal parathyroid glands in patients undergoing thyroid operations. Surgery 124, 1005-10 (1998).
[16]. Kuriloff, D. B. & Kizhner, V. Parathyroid gland preservation and selective autotransplantation utilizing topical lidocaine in total thyroidectomy. Laryngoscope 120, 1342-1344 (2010).
[17]. Ezzat, W. et al. Intraoperative parathyroid hormone as an indicator for parathyroid gland preservation in thyroid surgery. Swiss Med. Wkly. (2011). doi: 10.4414/smw.2011.13299
[18]. Fortuny, J. V. et al. Parathyroid gland angiography with indocyanine green fluorescence to predict parathyroid function after thyroid surgery. Br. J. Surg. 103, 537-543 (2016).
[19]. Boas, D. A. & Dunn, A. K. Laser speckle contrast imaging in biomedical optics. J. Biomed. Opt. 15, 11109 (2010).
[20]. Choi, B., Kang, N. M. & Nelson, J. S. Laser speckle imaging for monitoring blood flow dynamics in the in vivo rodent dorsal skin fold model. Microvasc. Res. 68, 143-6 (2004).
[21]. Iredahl, F., Löfberg, A., Sjöberg, F., Farnebo, S. & Tesselaar, E. Non-Invasive Measurement of Skin Microvascular Response during Pharmacological and Physiological Provocations. PLoS One 10, e0133760 (2015).
[22]. Dunn, A. K., Bolay, H., Moskowitz, M. A. & Boas, D. A. Dynamic Imaging of Cerebral Blood Flow Using Laser Speckle. J. Cereb. Blood Flow Metab. 21, 195-201 (2001).
[23]. Huang, Y. C., Ringold, T. L., Nelson, J. S. & Choi, B. Noninvasive blood flow imaging for real-time feedback during laser therapy of port wine stain birthmarks. Lasers Surg. Med. 40, 167-173 (2008).
[24]. Mirdell, R., Iredahl, F., Sj?berg, F., Farnebo, S. & Tesselaar, E. Microvascular blood flow in scalds in children and its relation to duration of wound healing: A study using laser speckle contrast imaging. Burns 42, 648-654 (2016).
[25]. Richards, L. M. et al. Intraoperative multi-exposure speckle imaging of cerebral blood flow. J. Cereb. Blood Flow Metab. 0271678X1668698 (2017). doi: 10.1177/0271678X16686987
[26]. Sexton, K. J., Zhao, Y., Davis, S. C., Jiang, S. & Pogue, B. W. Optimization of fluorescent imaging in the operating room through pulsed acquisition and gating to ambient background cycling. Biomed. Opt. Express 8, 2635 (2017).
[27]. Kirkpatrick, S. J., Duncan, D. D. & Wells-Gray, E. M. Detrimental effects of speckle-pixel size matching in laser speckle contrast imaging. Opt. Lett. 33, 2886 (2008).
[28]. Briers, D. et al. Laser speckle contrast imaging: theoretical and practical limitations. J. Biomed. Opt. 18, 66018 (2013).

What is claimed is:

1. A system for assessment of a tissue of a living subject, comprising:
a light source for emitting a beam of light;
a light delivery member coupled to the light source for delivering the beam of light onto a tissue surface of the living subject to illuminate the tissue surface;
a light collection member configured to collect light from the illuminated tissue surface responsive to the illumination;
a detector coupled to the collection member for acquiring images of the light from the illuminated tissue surface, wherein each of the acquired images comprises a speckle pattern; and
a controller arranged to operate the detector to acquire the images of the light from the illuminated tissue surface, receive the acquired images from the detector, and process the acquired images for the intraoperative assessment of tissue viability.

2. The system of claim 1, wherein the light source comprises a laser.

3. The system of claim 2, wherein the light source comprises a near infrared laser.

4. The system of claim 1, wherein the light delivery member comprises at least one optical fiber optically coupled to the light source for delivering the beam of light emitted from the light source.

5. The system of claim 4, wherein a diffuser is placed at a distal end of the at least one optical fiber for diffusing the beam of light onto the tissue surface.

6. The system of claim 5, wherein the diffuser is a hollow center diffuser.

7. The system of claim 4, wherein a beam shaper is placed at a distal end of the at least one optical fiber for focusing or collimating or directing the beam of light onto the tissue surface.

8. The system of claim 7, wherein the beam shaper comprises a biconvex lens.

9. The system of claim 8, wherein the beam shaper comprises a ball lens.

10. The system of claim 4, wherein the light delivery member further comprises a linear polarizer placed at the front of optic.

11. The system of claim 1, wherein the collection member comprises one or more optical fibers, including a fiber bundle.

12. The system of claim 11, wherein the collection member further comprises a rod lens placed on a distal end of the fiber(s) to optimize the collection of light and image formation at a predetermined working distance.

13. The system of claim 11, wherein the collection member further comprises a polarizer placed on a distal end of the fiber(s) for reduction of specular reflectance.

14. The system of claim 1, further comprising at least one lens placed in an optical path between the collection member and the detector for collecting and focusing light from the fiber(s) onto the detector.

15. The system of claim 1, wherein the detector comprises at least one camera.

16. The system of claim 15, wherein the at least one camera comprises a near-infrared camera.

17. The system of claim 15, wherein the at least one camera comprises a charge-coupled device (CCD) camera or a complementary metal oxide semiconductor (CMOS) camera.

18. The system of claim 1, wherein in the speckle contrast images, a perfused tissue has low speckle contrast, and a devascularized tissue has high speckle contrast.

19. The system of claim 1, further comprising a display for displaying the speckle contrast images of the tissue in real-time.

20. The system of claim 1, wherein the tissue is a parathyroid gland, a pancreas gland, or an adrenal gland.

21. A system for assessment of a tissue of a living subject, comprising:
a light source for emitting a beam of light to illuminate the tissue; and
an imaging head positioned over the target of interest for acquiring laser speckle contrast imaging (LSCI) images of light from the illuminated tissue responsive to the illumination.

22. The system of claim 21, wherein the light source comprises a near infrared laser.

23. The system of claim 21, wherein the imaging head comprises:
an illumination channel coupled to the light source for delivering the beam of light onto the tissue surface to illuminate the tissue surface; and
an imaging channel positioned in relationship to the illumination channel for acquiring images of the light from the illuminated tissue surface, wherein each of the acquired images comprises a speckle pattern.

24. The system of claim 21, wherein the light source is placed in the illumination channel.

25. The system of claim 21, wherein a diffuser is placed at a distal end of the illumination channel for diffusing the beam of light onto the tissue surface.

26. The system of claim 21, wherein a beam shaper is placed at a distal end of the illumination channel for focusing or collimating or directing the beam of light onto the tissue surface.

27. The system of claim 26, wherein the beam shaper comprises a ball lens.

28. The system of claim 23, wherein the illumination channel comprises a linear polarizer placed at the front of the beam delivery system.

29. The system of claim 18, wherein the imaging channel comprises at least one camera.

30. The system of claim 29, wherein the at least one camera comprises a charge-coupled device (CCD) camera or a complementary metal oxide semiconductor (CMOS) camera.

31. The system of claim 29, wherein the imaging channel further comprises a polarizer placed the front of the camera for reduction of specular reflectance.

32. The system of claim 23, further comprises a controller configured to control operations of the system for acquiring the LSCI images of the illuminated tissue, receiving the acquired LSCI images from the at least one camera, and processing the acquired LSCI images to obtain speckle contrast images for the intraoperative assessment of tissue viability.

33. The system of claim 23, wherein the tissue is a parathyroid gland, a pancreas gland, or an adrenal gland.

34. A method for assessment of a tissue of a living subject, comprising:
directing a beam of light onto a tissue surface of the target of interest to illuminate the tissue surface;
acquiring laser speckle contrast imaging (LSCI) images of light from the illuminated target of interest responsive to the illumination; and
processing the acquired LSCI images for the intraoperative assessment of tissue viability.

35. The method of claim 34, wherein said processing the acquired images comprises calculating a plurality of speckle contrasts from the acquired images of the target of interest.

36. The method of claim 35, wherein said calculating plurality of speckle contrasts comprises:
defining a window with a number of pixels over which a speckle contrast is to be calculated;
moving the window across the acquired image of the speckle pattern; and
at each location, calculating the speckle contrast as a standard deviation of pixel intensity values σₛ within the window divided by a mean intensity value [symbol] I [symbol] as follows:
$${K}_{s} = \frac{{σ}_{s}}{〈 I 〉}$$
wherein the resultant speckle contrast image has values that range from 0 to 1, with values closer to 0 representing regions of greater motion (perfusion) and 1 representing regions with no motion.

37. The method of claim 34, wherein said processing of the acquired images comprises an LSCI threshold mechanism for frame selection that removes frames that contain excessive motion, thereby realizing the intraoperative assessment of tissue viability.

38. The method of claim 37, wherein a frame selection code is applied to selection frames with tolerable motion.

39. The method of claim 37, wherein a universal mean of the contrast value over each frame is the threshold with a calculated number based on the acquired video and threshold applied, such that when a frame is below the threshold, there exists excessive amount of motion artifact that confounds with the blood flow information in the frame; and when a frame is above the threshold, the frame is acquired in relative stillness and the motion artifact that is introduced during the exposure time is negligible for speckle contrast calculation.

40. The method of claim 39, wherein said processing of the acquired images comprises discarding a frame out of the video when the frame is below the threshold, and preserving a frame when the frame is above the threshold and using the preserved frame an indicator of whether the target of interest is well vascularized.

41. The method of claim 37, wherein a machine learning algorithm is applied to reduce motion artifact on the acquired LSCI images.

42. The method of claim 34, further comprising for displaying the speckle contrast images of the parathyroid gland in real-time.
